(19) United States
(12) Patent Application Publication
(10) Pub. No.: US 2011/0223635 A1
(43) Pub. Date: Sep. 15, 2011

(54) Method and Composition for Controlling Gene Expression

(76) Inventors: Karl Deisseroth, Palo Alto, CA (US); Feng Zhang, Palo Alto, CA (US)

(21) Appl. No.: 13/057,695

(22) PCT Filed: Aug. 11, 2009
(86) PCT No.: PCT/US09/53474

§ 371 Date: Apr. 29, 2011

Related U.S. Application Data
(60) Provisional application No. 61/087,903, filed on Aug. 11, 2008.

(51) Int. Cl. C12P 21/00; C12N 15/85; C12N 5/071

(52) U.S. Cl. 435/69.1; 435/320.1; 435/325

(57) ABSTRACT

A composition for expressing a protein in cells is provided. In certain embodiments, a circular expression vector provided herein comprises: a promoter, a coding sequence encoding a protein of interest, in which the coding sequence is in a reversed 3′-5′ orientation, a transcription termination sequence, and at least a first recombination site and a second recombination site flanking the coding sequence. A method for using the disclosed composition and a kit comprising the composition are also provided herein.

### CROSS-REFERENCE

This application claims the benefit of U.S. Provisional Application No. 61/087,903, filed Aug. 11, 2008, which application is incorporated by reference herein in its entirety.

### BACKGROUND OF THE INVENTION

Genetic tools that enable researchers to control gene expression, to delete undesired DNA sequences, and to modify chromosome architecture have been indispensable in the advancement of biotechnology discoveries and biomedical applications.

Precise temporal and spatial control of protein expression in a cell or in an organism opens the door to many opportunities in the studies of both gene expression and the physiological effects of transgenes. As such, the ability to control the site of integration, the number of integrated copies and the level of expression of transgenes is very important and requires efficient and reliable genetic tools.

In many applications, the success in regulating protein expression depends largely on the ability to achieve a combination of stable chromosomal integration and a tight control over the expression of transferred genes. Certain applications utilize drug-inducible systems and various genetic regulatory elements in the control of gene expression in cells or transgenic animals. By alleviating problems commonly encountered in these systems such as leakiness, insufficient levels of induction, and lack of tissue specificity, one may increase the in vivo functionality of these genetic tools.

The present invention addresses these needs.

### SUMMARY OF THE INVENTION

A composition for expressing a protein in cells is provided. In certain embodiments, a circular expression vector provided herein comprises: a promoter, a coding sequence encoding a protein of interest, in which the coding sequence is in a reversed 3′-5′ orientation, a transcription termination sequence, and at least a first recombination site and a second recombination site flanking the coding sequence. A method for using the disclosed composition and a kit comprising the composition are also provided herein.

These and other objects, advantages, and features of the invention will become apparent to those persons skilled in the art upon reading the details of the composition as more fully described below.

### BRIEF DESCRIPTION OF THE DRAWINGS

This disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1A schematically illustrates certain features of one embodiment of the composition provided herein. FIG. 1B schematically illustrates certain features of another embodiment of the composition provided herein with a multiple cloning site. FIG. 1C schematically illustrated certain features of an additional embodiment of the composition provided herein. FIG. 1D schematically illustrated certain features of an additional embodiment of the composition with a multiple cloning site.

FIG. 2A schematically illustrates certain examples of expression cassettes. FIG. 2B presents fluorescence micrographs of HEK293 cells in the presence or absence of Cre recombinase, as indicated. Cells in the top panels were transfected with the Floxed Stop construct. Cells in the middle panels were transfected with the Single-floxed Reverse ORF (SIO) construct. Cells in the bottoms panels were transfected with the Double-floxed Reverse ORF (DIO) construct. Results from the FACS analysis of the various cell populations in FIG. 2B are represented as normalized cell population versus raw YFP fluorescence (FIG. 2C). FIG. 2D schematically illustrates certain features of the composition and method provided herein with an exemplary DIO construct.

FIG. 3A presents fluorescence micrographs of hippocampal neurons transfected with DIO constructs containing a coding region for the protein ChR2-EYFP. Middle panel monitors the presence of parvalbumin (PV) and the right panel overlays Chr2-EYFP with PV. Percentage of cells that are positive for either YFP or PV are graphed in FIG. 3B. Current trace of a neuron transfected with Chr2-EYFP construct when subjected to a light stimulus is shown in FIG. 3C. Voltage trace of a neuron transfected with the Chr2-EYFP construct when subjected to pulses of light stimulus is shown in FIG. 3D.

FIG. 4A depicts several exemplary microbial opsins that may be used in the subject method and their electrophysiological activities in response to light.

FIG. 4B depicts exemplary strategies in utilizing anterograde and retrograde transport for selective gene expression.

FIG. 5A is a schematic illustrating gene expression targeting using anterograde transport. FIG. 5B is a schematic illustrating gene expression targeting using retrograde transport. FIG. 5C depicts construct design for the WGA-CRE and Cre-TTC adeno-associated virus vectors used in FIGS. 5A and 5B, respectively. FIG. 5D shows fluorescence images demonstrating the activation of Cre-dependent gene expression in the contralateral dentate gyms and the Cre-expression of the virus vectors in the ipsilateral dentate gyms.

FIG. 6 summarizes nine exemplary strategies for controlling a specific neural component in one of the nine given networks, each of which comprises cell population A, cell population B, and cell population C.

### DEFINITIONS

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A polynucleotide is typically composed of a specific sequence of four nucleotide bases: adenine (A); cytosine (C); guanine (G); and thymine (T), (uracil (U) for thymine (T) when the polynucleotide is RNA). Thus, the term polynucleotide sequence is the alphabetical representation of a polynucleotide molecule. This alphabetical representation can be input into databases in a computer having a central processing unit and used for bioinformatics applications such as functional genomics and homology searching.

A “polypeptide” is a sequence or a portion thereof that contains an amino acid sequence of at least 3 to 5 amino acids, more preferably at least 8 to 10 amino acids, and even more preferably at least 15 to 20 amino acids. A polypeptide may be encoded by a nucleic acid sequence. Also encompassed are polypeptide sequences that are immunologically identifiable with a polypeptide encoded by the sequence.

A “coding sequence” or a sequence that “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding such regulatory polypeptides, that may optionally include intron sequences derived from chromosomal DNA.

As used here, the term “inverted” or “inversion” refers to a nucleic acid sequence that is in an opposite orientation specified by its 5′ and 3′ ends relative to its original orientation in the context of a genome or a longer polynucleotide. In certain embodiments, 3′ to 5′ coding sequence may be referred herein as in the “reversed 3′ to 5′ orientation” due to the convention of annotating nucleic acid elements in the 5′ to 3′ direction.

The term “promoter” or “promoter element” is defined herein as a nucleic acid that directs transcription of a downstream polynucleotide in a cell. In certain cases, the polynucleotide may contain a coding sequence and the promoter may direct the transcription of the coding sequence into translatable RNA.

“Operably linked” refers to an arrangement of elements wherein the components so described are configured so as to perform their usual function. Thus, a given promoter that is operably linked to a coding sequence (e.g., a reporter expression cassette) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct,” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as integrating vectors.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence.

An “expression cassette” comprises any nucleic acid construct capable of directing the expression of a gene/coding sequence of interest. Such cassettes can be constructed into a “vector,” “vector construct,” “expression vector,” or “gene transfer vector,” in order to transfer the expression cassette into target cells. Thus, the term includes cloning and expression vehicles, as well as viral vectors. Expression cassettes include at least promoters and optionally, transcription termination signals. Additional factors necessary or helpful in effecting expression can also be used as described herein. For example, transcription termination signals, enhancers, and other nucleic acid sequences that influence gene expression, can also be included in an expression cassette.

The term “episomal vector,” as used herein, refers to a vector introduced into the target cells that does not integrate into, i.e., insert into, the target cell genome, i.e., one or more chromosomes of the target cell. In other words, an episomal vector does not fuse with or become covalently attached to chromosomes present in the target cell into which it is introduced. Accordingly, episomal vectors provide for persistent expression, while being maintained episomally.

The term “exogenous” is defined herein as DNA which is introduced into a cell. Exogenous DNA can possess sequences identical to or different from the endogenous DNA present in the cell prior to transfection.

As used herein, “transgene” or “transgenic element” refers to an artificially introduced, chromosomally integrated nucleic acid sequence present in the genome of a host organism.

The term “transgenic animal” means a non-human animal having a transgenic element integrated in the genome of one or more cells of the animal. “Transgenic animals” as used herein thus encompasses animals having all or nearly all cells containing a genetic modification (e.g., fully transgenic animals, particularly transgenic animals having a heritable transgene) as well as chimeric, transgenic animals, in which a subset of cells of the animal are modified to contain the genomically integrated transgene.

“Target cell” as used herein refers to a cell that in which a genetic modification is desired. Target cells can be isolated (e.g., in culture) or in a multicellular organism (e.g., in a blastocyst, in a fetus, in a postnatal animal, and the like). Target cells of particular interest in the present application include, but not limited to, cultured mammalian cells, including CHO cells, and stem cells (e.g., embryonic stem cells (e.g., cells having an embryonic stem cell phenotype), adult stem cells, pluripotent stem cells, hematopoietic stem cells, mesenchymal stem cells, and the like).

“Recombinases” are a family of enzymes that mediate site-specific recombination between specific DNA sequences recognized by the recombinase (Esposito, D., and Scocca, J. J., Nucleic Acids Research 25, 3605-3614 (1997); Nunes-Duby, S. E., et al., Nucleic Acids Research 26, 391-406 (1998); Stark, W. M., et al., Trends in Genetics 8, 432-439 (1992)). Within this group are several subfamilies including “Integrase” or tyrosine recombinase (including, for example, Cre and λ integrase) and “Resolvase/Invertase” or serine recombinase (including, for example, φC31 integrase, R4 integrase, and TP-901 integrase). The term also includes recombinases that are altered as compared to wild-type, for example as described in U.S. Patent Publication 20020094516, the disclosure of which is hereby incorporated by reference in its entirety herein.

A “unidirectional site-specific recombinase” is a naturally-occurring recombinase, such as the φC31 integrase, a mutated or altered recombinase, such as a mutated or altered φC31 integrase that retains unidirectional, site-specific recombination activity, or a bi-directional recombinase modified so as to be unidirectional, such as a Cre recombinase that has been modified to become unidirectional.

“Site-specific integration” or “site-specifically integrating” as used herein refers to the sequence specific recombination and integration of a first nucleic acid with a second nucleic acid, typically mediated by a recombinase. In general, site-specific recombination or integration occurs at particular defined sequences recognized by the recombinase. These defined sequences are referred herein as “recombination sites.” In contrast to random integration, site specific integration occurs at a particular sequence at a higher efficiency.

The term “recombination site,” as used herein, refers to a nucleotide sequence recognized by a recombinase so that a recombination event may occur. In certain cases, the recombination sites may be recognizable by Cre, Flp, integrase, or other recombinase. An example of a recombination site recognizable by the Cre recombinase is loxP, a sequence of about 34 base pairs comprising an ˜8 base pair core sequence flanked by two ˜13 base pair inverted repeats (Sauer, Curr. Opin. Biotech. 5:521-527, 1994). In certain embodiments, the recombination sites are sequences between short (about 15 to about 40 base pair) Flipase Recognition Target (FRT) sites, recognizable by the Flipase recombination enzyme (FLP or Flp) derived from the yeast Saccharomyces cerevisiae (U.S. Pat. No. 5,527,695, Lyznik et al. Nucleic Acid Res. 24:3784-3789, 1996, and O'Gorman et al., Science, 251:1351-1355, 1991). Another recombination sites may be attP or attB attachment site sequences that are recognizable by phage φC31.

A “native recognition site”, as used herein, means a recognition site that occurs naturally in the genome of a cell (i.e., the sites are not introduced into the genome, for example, by recombinant means).

A “pseudo-site” or a “pseudo-recombination site” as used herein means a DNA sequence comprising a recognition site that is bound by a recombinase enzyme where the recognition site differs in one or more nucleotides from a wild-type recombinase recognition sequence and/or is present as an endogenous sequence in a genome that differs from the sequence of a genome where the wild-type recognition sequence for the recombinase resides. For a given recombinase, a pseudo-recombination sequence is functionally equivalent to a wild-type recombination sequence, occurs in an organism other than that in which the recombinase is found in nature, and may have sequence variation relative to the wild type recombination sequences. In some embodiments a “pseudo attP site” or “pseudo attB site” refer to pseudo sites that are similar to the recognitions site for wild-type phage (attP) or bacterial (attB) attachment site sequences, respectively, for phage integrase enzymes, such as the phage φC31.

Methods of transfecting cells are well known in the art. By “transfected” it is meant an alteration in a cell resulting from the uptake of foreign nucleic acid, usually DNA. Use of the term “transfection” is not intended to limit introduction of the foreign nucleic acid to any particular method. Suitable methods include viral infection, conjugation, electroporation, particle gun technology, calcium phosphate precipitation, direct microinjection, and the like. The choice of method is generally dependent on the type of cell being transfected and the circumstances under which the transfection is taking place (i.e. in vitro, ex vivo, or in vivo). A general discussion of these methods can be found in Ausubel, et al, Short Protocols in Molecular Biology, 3rd ed., Wiley & Sons, 1995.

### DETAILED DESCRIPTION OF THE INVENTION

A composition for expressing a protein in cells is provided. In certain embodiments, a circular expression vector provided herein comprises: a promoter, a coding sequence encoding a protein of interest, in which the coding sequence is in a reversed 3′-5′ orientation, a transcription termination sequence, and at least a first recombination site and a second recombination site flanking the coding sequence.

Before the present composition are described, it is to be understood that this invention is not limited to particular composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a vector” includes a plurality of such vectors and reference to “the protein” includes reference to one or more proteins and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Compositions

As noted above, in certain embodiments, the composition provided herein comprises a circular expression vector comprising: a promoter, a coding sequence encoding a protein of interest, in which the coding sequence is in a reversed 3′-5′ orientation, a transcription termination sequence, and at least a first recombination site and a second recombination site flanking the coding sequence.

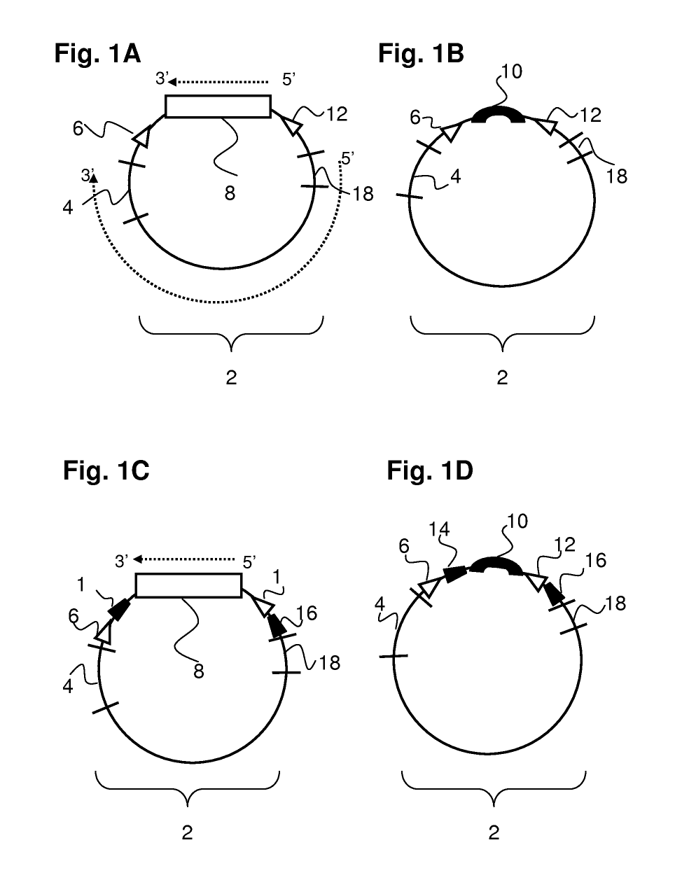

Certain features of the subject composition are illustrated in FIG. 1 and are described in greater detail below. With reference to FIG. 1, the circular expression vector 2 comprises the following elements in order from 5′ to 3′, a promoter sequence 4, a first recombination site 6, a coding sequence 8, a second recombination site 12, and a transcription termination sequence 18.

In certain embodiments, the coding sequence is oriented in the reversed 3′ to 5′, in contrast to the rest of the nucleic acid elements in the vector. In other words, the coding sequence is positioned in the antisense orientation with regard to the promoter. This reversed position prevents the expression of the coding sequence in the presence of a functional machinery for transcription or translation because the sequence is not in the correct orientation for transcription or translation to proceed. For example, a transcript made from such reversed coding sequence may lack a proper start codon or may not in the right frame, giving rise to a premature stop codon.

Generally, the coding sequence is positioned in an orientation such that a product would express only if the orientation is inverted. In certain embodiments, the first and second recombination sites flanking the coding sequence are recognized by a recombinase that causes the coding sequence to undergo recombination, and consequently inverting the orientation of the coding sequence. A feature of the invention is that the product resulting from a recombination reaction is incapable of undergoing subsequence recombination. Hence, once the coding sequence in the reversed 3′ to 5′ orientation undergoes a recombination, the sequence becomes inverted and stays in the 5′ to 3′ orientation for the lifetime of the vector. The fact that the product of the recombination reaction is incapable of further recombination means that the recombined vector permanently comprises a coding sequence that is capable of being expressed. The coding sequence would not be excised out or recombined back into the reversed 3′ to 5′ position.

In certain embodiments, with reference to FIG. 1, a first and a second recombination sites (6 and 12) may be a phage attachment site (“attP”) and a bacterial attachment site (“attB”), respectively. The native attB and attP recognition sites of phage φC31 (i.e. bacteriophage φC31) are generally about 34 to 40 nucleotides in length (Groth et al. Proc Natl Acad Sci USA 97:5995-6000 (2000)). These sites are typically arranged as follows: attP comprises a first DNA sequence (attP5′), a core region, and a second DNA sequence (attP3′) in the relative order attP5′-core region-attP3′; AttB comprises a first DNA sequence attB5′, a core region, and a second DNA sequence attB3′ in the relative order attB5′-core region-attB3′.

For example, for the phage φC31 attP (the phage attachment site), the core region is 5′-TTG-3′ the flanking sequences on either side are represented here as attP5′ and attP3′, the structure of the attP recombination site is, accordingly, attP51-TTG-attP3′. Correspondingly, for the native bacterial genomic target site (attB) the core region is 5′-TTG-3′, and the flanking sequences on either side are represented here as attB5 ‘ and attB3′, the structure of the attB recombination site is, accordingly, attB51-TTG-attB3’.

Because the attB and attP sites are different sequences, recombination results in a hybrid site-specific recombination site (designated attL or attR for left and right) that is neither an attB sequence or an attP sequence, and is functionally unrecognizable as a site-specific recombination site (e.g., attB or attP) to the relevant unidirectional site-specific recombinase, thus removing the possibility that the unidirectional site-specific recombinase will catalyze a second recombination reaction between the attL and the attR, reversing the first recombination reaction. For example, after a single-site, φC31 integrase-mediated, recombination event takes place the result is the following recombination product: attB5′-TTG-attP3′{φC31 vector sequences}attP5′-TTG-attB3′. Typically, after recombination the post-recombination recombination sites are no longer able to act as substrate for the φC31 recombinase. This results in stable integration with little or no recombinase mediated excision.

In certain cases, the recombination sites in the subject vector may be native recombination sites, found to exist in the genomes of a variety of organisms. The native recombination site does not necessarily have a nucleotide sequence identical to the wild-type recombination sequences (for a given recombinase); but such native recombination sites are nonetheless sufficient to promote recombination meditated by the recombinase. Such recombination site sequences are referred to herein as “pseudo-recombination sequences.” For a given recombinase, a pseudo-recombination sequence is functionally equivalent to a wild-type recombination sequence, occurs in an organism other than that in which the recombinase is found in nature, and may have sequence variation relative to the wild type recombination sequences.

Identification of pseudo-recombination sequences can be accomplished, for example, by using sequence alignment and analysis, where the query sequence is the recombination site of interest (for example, attP and/or attB).

In general, the unidirectional site-specific integrase interaction with the site-specific recombination sites produces a recombination product that does not contain a sequence that acts as an effective substrate for the unidirectional site-specific integrase. Thus, the recombination event occurring with the subject composition is unidirectional, with little or no detectable excision of the introduced nucleic acid mediated by the unidirectional site-specific integrase.

In addition, to a first and a second recombination sites, in certain embodiments, the subject composition may further comprise a third recombination site interposed between the first recombination site and the coding sequence and a fourth recombination site interposed between the second recombination site and the transcription termination region, wherein the first recombination site and the second recombination site recombine in the presence of a recombinase and the third recombination site and the fourth recombination site recombine in the presence of a recombinase.

With reference to FIG. 1C, the subject composition according to the above embodiment is an expression vector comprising, in an order from 5′ to 3′: a promoter 4, a first recombination site 6, a third recombination site 14, a coding sequence in a reversed 3′ to 5′ orientation 8, a second recombination site 12, a fourth recombination site 16, followed by a transcription termination sequence 18.

A vector where there are two recombination sites flanking each side of the coding sequence may undergo a recombination event to invert the coding sequence into a 5′ to 3′ orientation to turn on expression. After such a recombination event, two of the four existing recombination sites may be excised in a subsequent recombination to produce a vector with one recombination site flanking each side of the coding sequence. This two-step recombination event produces a vector that is incapable of subsequent recombination and the coding sequence remains in the 5′ to 3′ orientation.

In certain cases, with reference to FIG. 1C, the first recombination site 6 and the second recombination site 12 form a first pair of compatible sites such that a recombinase would recognize the first and second sites to catalyze a recombination event. Accordingly, the third and the fourth recombination sites (14 and 16) form a second pair of compatible sites such that a recombination event may occur based on the recognition of these compatible sites by a recombinase. In certain embodiments, a recombination site from a first pair is not compatible with either site in a second pair. A recombinase is not able to catalyze a recombination event to excise the coding sequence or to invert the coding sequence if the only recombination sites flanking the coding sequence are incompatible.

As such, in an embodiment where a vector comprises four recombination sites, as described above, the subject vector may lose compatible recombination sites after a two-step recombination event, rendering the coding sequence incapable of subsequence recombination.

In certain cases, the recombination sites in the subject vector may be recognizable by a Cre, Flp, or other recombinase. For example, the recognition sequence for Cre recombinase is loxP which is a sequence of about 34 base pairs comprising an 8 base pair core sequence flanked by two 13 base pair inverted repeats (serving as the recombinase binding sites) (Sauer, Curr. Opin. Biotech. 5:521-527, 1994). In other embodiments, the recognition sequence for Cre recombinase is lox2722, which contain certain mutations that render the site incompatible with loxP. Other incompatible recombination sites may also be used.

In certain embodiments, the recombination sites are sequences between short (about 15 to about 40 base pair) Flipase Recognition Target (FRT) sites, recognizable by the Flipase recombination enzyme (FLP or Flp) derived from the yeast Saccharomyces cerevisiae (U.S. Pat. No. 5,527,695, Lyznik et al. Nucleic Acid Res. 24:3784-3789, 1996, and O'Gorman et al., Science, 251:1351-1355, 1991).

Other examples of recognition sequences are the attB, attP, attL, and attR sequences, as described above, which are recognized by the recombinase enzyme λ Integrase. AttB is an approximately 25 base pair sequence containing two 9 base pair core-type Int binding sites and a 7 base pair overlap region. AttP is an approximately 240 base pair sequence containing core-type Int binding sites and arm-type Int binding sites as well as sites for auxiliary proteins IHF, FIS, and Xis. See Landy, Curr. Opin. Biotech. 3:699-707 (1993). Other phage integrases (such as the R4 phage integrase) and their recognition sequences can be adapted for use in the invention.

In certain cases, a pair of compatible loxP sites is positioned in the reversed orientation with respect to one another so that recombination between the two sites within the same polynucleotide leads to an inversion of the intervening sequences, as opposed to excision. For example, sites 6 and 12 in FIG. 1 may be compatible loxP sites that are positioned in the reversed orientation with respect to one another. Recombination between site 6 and 12 leads to inversion of the coding sequence 8. In certain embodiments, the inversion of the coding sequence leads to one pair of compatible recombination sites on one side of the coding sequence in the same orientation, as opposed to the reversed orientation with respect to one another. Such position of the recombination sites allows subsequent excision of the intervening sequence, leaving the coding sequence flanked by incompatible sites. Accordingly, the coding sequence may not undergo another recombination event due to the absence of compatible recombination sites.

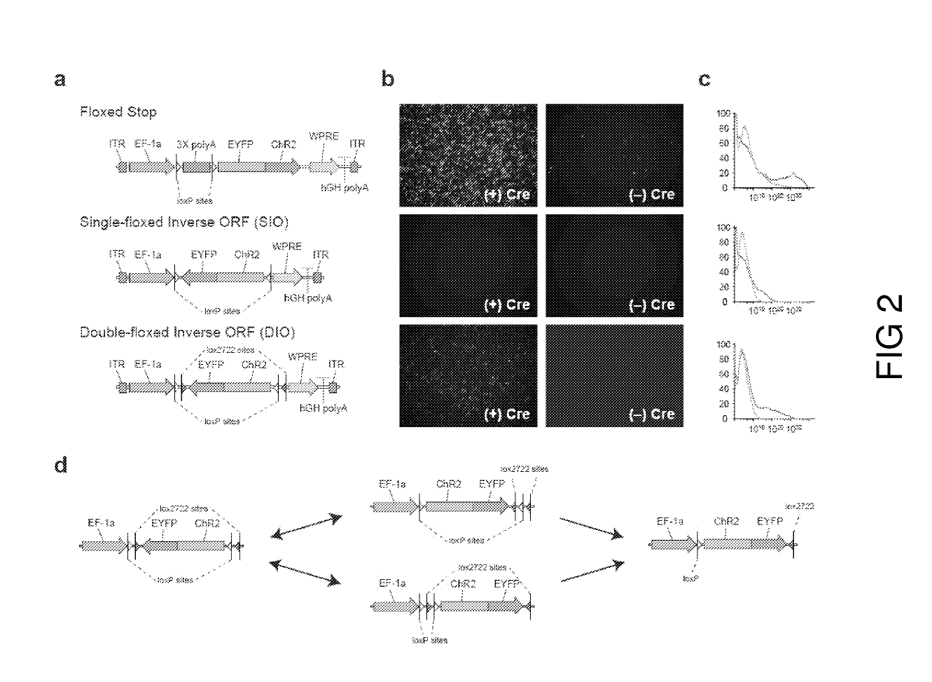

Certain features of this embodiment are illustrated in an exemplary expression cassette in FIG. 2D. The expression cassette may be present in the context of a larger polynucleotide, such as a vector of the subject invention. In an order from 5′ to 3′, the cassette contains a promoter, such as EF-1a, one loxP site, one lox2722 site, followed by an exemplary coding sequence labeled as ChR2-EYFP in a reversed orientation, and a loxP site, followed by another lox2722 site. Following a recombination event, the coding sequence is inverted to be in an orientation enabling correct transcription and translation. One pair of compatible recombination sites are also rearranged to be either on the 5′ side or the 3′ side of ChR2-EYFP, depending on which pair of recombination sites is used by the recombinase. For example, in the middle top panel, both lox2722 sites are located on the 3′ side of ChR2-EYFP, as directed repeats, as opposed to the inverted repeats that have existed before the recombination event. A different scenario is illustrated by the bottom panel, where both loxP sites may be located on the 5′ side of ChR2-EYFP, also as directed repeats. The orientation of the directed repeats leads to an excision of the intervening sequence by the recombinase. The final product is an expression cassette in the right panel containing: in an order from 5′ to 3′, a promoter EF-1a, a loxP site, ChR2-EYFP, followed by a lox2722 site. Since loxP and lox2722 are incompatible with each other, their intervening sequence, namely the coding sequence ChR2-EYFP, is incapable of subsequent recombination.

This feature of the subject composition ensures stability of expression of proteins of interest once a recombination event has taken place to invert the coding sequence to a translatable 5′ to 3′ orientation.

In certain embodiments, the subject composition may also comprise selectable markers, an origin of replication, and other elements such as an inducible element sequence, an epitope tag sequence, a promoter, or promoter-enhancer sequences, and the like. See, e.g., U.S. Pat. No. 6,632,672, the disclosure of which is incorporated by reference herein in its entirety. The promoter element is discussed in more detail below.

The promoter sequence is operably linked to the coding sequence as to promote the transcription of the coding sequence when the appropriate enzymes are present. Promoter and promoter-enhancer sequences are DNA sequences to which RNA polymerase binds and initiates transcription. The promoter determines the polarity of the transcript by specifying which strand will be transcribed. Bacterial promoters consist of consensus sequences, −35 and −10 nucleotides relative to the transcriptional start, which are bound by a specific sigma factor and RNA polymerase.

Eukaryotic promoters are more complex. Most eukaryotic promoters utilized in expression vectors are transcribed by RNA polymerase II. General transcription factors (GTFS) first bind specific sequences near the transcription start site and then recruit the binding of RNA polymerase II. In addition to these minimal promoter elements, small sequence elements are recognized specifically by modular DNA-binding, trans-activating proteins (e.g. AP-1, SP-1) that regulate the activity of a given promoter. Viral promoters serve the same function as bacterial or eukaryotic promoters and either require a promoter-specific RNA polymerase in trans (e.g., bacteriophage T7 RNA polymerase in bacteria) or recruit cellular factors and RNA polymerase II (in eukaryotic cells). Viral promoters (e.g., the SV40, RSV, and CMV promoters) may be preferred as they are generally particularly strong promoters.

Promoters may be, furthermore, either constitutive or regulatable. Constitutive promoters constantly express the gene of interest. In contrast, regulatable promoters (i.e., derepressible or inducible) express genes of interest only under certain conditions that can be controlled. Derepressible elements are DNA sequence elements which act in conjunction with promoters and bind repressors (e.g. lacO/lacIq repressor system in E. coli). Inducible elements are DNA sequence elements which act in conjunction with promoters and bind inducers (e.g. gal1/gal4 inducer system in yeast). In either case, transcription is virtually “shut off” until the promoter is derepressed or induced by alteration of a condition in the environment (e.g., addition of IPTG to the lacO/lacIq system or addition of galactose to the gal1/gal4 system), at which point transcription is “turned-on.”

Another type of regulated promoter is a “repressible” one in which a gene is expressed initially and can then be turned off by altering an environmental condition. In repressible systems transcription is constitutively on until the repressor binds a small regulatory molecule at which point transcription is “turned off”. An example of this type of promoter is the tetracycline/tetracycline repressor system. In this system when tetracycline binds to the tetracycline repressor, the repressor binds to a DNA element in the promoter and turns off gene expression.

Examples of constitutive prokaryotic promoters include the int promoter of bacteriophage λ, the bla promoter of the β-lactamase gene sequence of pBR322, the CAT promoter of the chloramphenicol acetyl transferase gene sequence of pPR325, and the like.

Examples of inducible prokaryotic promoters include the major right and left promoters of bacteriophage (P_{L} and P_{R}), the trp, recA, lacZ, AraC and gal promoters of E. coli, the α-amylase (Ulmanen Ett at., J. Bacteriol. 162:176-182, 1985) and the sigma-28-specific promoters of B. subtilis (Gilman et al., Gene sequence 32:11-20 (1984)), the promoters of the bacteriophages of Bacillus (Gryczan, In: The Molecular Biology of the Bacilli, Academic Press, Inc., NY (1982)), Streptomyces promoters (Ward et at., Mol. Gen. Genet. 203:468-478, 1986), and the like. Exemplary prokaryotic promoters are reviewed by Glick (J. Ind. Microtiot. 1:277-282, 1987); Cenatiempo (Biochimie 68:505-516, 1986); and Gottesman (Ann. Rev. Genet. 18:415-442, 1984).

Exemplary constitutive eukaryotic promoters include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gal1 gene sequence promoter (Johnston et al., Proc. Natl. Acad. Sci. USA 79:6971-6975, 1982); Silver et al., Proc. Natl. Acad. Sci. USA 81:5951-59SS, 1984), the CMV promoter, the EF-1 promoter.

Examples of inducible eukaryotic promoters include, but are not limited to, the following: ecdysone-responsive promoters, the tetracycline-responsive promoter, promoters regulated by “dimerizers” that bring two parts of a transcription factor together, estrogen-responsive promoters, progesterone-responsive promoters, riboswitch-regulated promoters, antibiotic-regulated promoters, acetaldehyde-regulated promoters, and the like.

Some regulated promoters can mediate both repression and activation. For example, in the RheoSwitch system a protein (the RheoReceptor) binds to a DNA element (UAS, upstream activating sequence) in the promoter and mediates repression. However in the presence of certain ecdysone-like inducers another protein (the RheoActivator) will bind to the inducer. The inducer-bound RheoActivator is capable of binding to the DNA-bound RheoReceptor. The RheoReceptor/inducer/RheoActivator is then capable of activating gene expression.

As noted above, in certain embodiments, the subject composition also comprises selectable markers. Common selectable marker genes include those for resistance to antibiotics such as ampicillin, tetracycline, kanamycin, bleomycin, streptomycin, hygromycin, neomycin, puromycin, G418, bleomycin, blasticidin, Zeocin™, and the like. Selectable auxotrophic genes include, for example, hisD, that allows growth in histidine free media in the presence of histidinol.

A further element useful in an expression vector is an origin of replication. Replication origins are unique DNA segments that contain multiple short repeated sequences that are recognized by multimeric origin-binding proteins and that play a key role in assembling DNA replication enzymes at the origin site. Suitable origins of replication for use in expression vectors employed herein include E. coli oriC, ColE1 plasmid origin, 2μ and ARS (both useful in yeast systems), sf1, SV40, EBV oriP (useful in eukaryotic systems, such as a mammalian system), and the like.

In certain cases, the subject vector is an episomal vector. In certain embodiments, the composition contains an Epstein Barr virus (EBV) oriP origin of replication, which permits episomal replication in cell lines expressing EBV nuclear antigen (EBNA-1). Vectors having the EBV origin and the nuclear antigen EBNA-1 are capable of replication to high copy number in mammalian cells without being integrated in the genome of the cell. In certain embodiments, the presence of EBNA-1 in combination with the OriP latent origin of replication, confer the functions of autonomous episomal replication and nuclear retention in a stable copy number, replicating only once per cell cycle.

In certain embodiments, the subject composition is derived from adenovirus or comprises adenovirus-associated components. For example, an episomal vector may be characterized by the following features: (i) an element, such as the EBV plasmid origin of replication, which renders the episome capable of autonomous replication and maintains the episome in multiple copies by promoting nuclear retention, (ii) an adenoviral type Ad5 inverted terminal repeat (ITRs) junction; and (iii) elements mediating the expression of adenoviral genes necessary for adenoviral replication (e.g., polymerase, pre-terminal protein and DNA binding protein, as well as early region 4 (E4) ORF6. Details of the adenoviral genomes and the methods of using adenovirus-associated vectors are discussed in U.S. Pat. Nos. 6,303,362 and 7,045,344, disclosures of which are incorporated herein by reference.

In certain embodiments, the subject composition further comprises an internal ribosome entry site (IRES) positioned in the coding sequence between the transcription start site and the translation initiation codon of the protein of interest. Such vectors may allow for increased gene expression if they are translational enhancers or they can also allow for production of multiple proteins of interest from a single transcript, as long as an IRES is located 5′ to each coding region of interest.

In certain cases, the subject composition includes a multiple cloning site or polylinker A multiple cloning site or polylinker is a synthetic DNA encoding a series of restriction endonuclease recognition sites inserted into the subject vector and allows for convenient cloning of polynucleotides of interest into the donor vector at a specific position. By “polynucleotide of interest” it is meant any nucleic acid fragment adapted for introduction into a target cell. Suitable examples of polynucleotides of interest include promoter elements, therapeutic genes, marker genes, control regions, trait-producing fragments, nucleic acid elements to encode a polypeptide, gene disruption elements, as well as nucleic acids that do not encode for a polypeptide, including a polynucleotide that encodes a non-translated RNA, such as a shRNA that may play a role in RNA interference (RNAi) based gene expression control. FIGS. 1B and 1D schematically illustrate certain features of an embodiment of the subject composition comprising a multiple cloning site 10. In certain cases, the multiple cloning site 10 enables the insertion of a coding sequence in the reversed 3′ to 5′ orientation relative to the promoter 4.

The subject composition described herein can be constructed utilizing methodologies known in the art of molecular biology (see, for example, Ausubel or Maniatis) in view of the teachings of the specification. An exemplary method of obtaining polynucleotides, including suitable regulatory sequences (e.g., promoters) is PCR. General procedures for PCR are taught in MacPherson et al., PCR: A PRACTICAL APPROACH, (IRL Press at Oxford University Press, (1991)). PCR conditions for each application reaction may be empirically determined A number of parameters influence the success of a reaction. Among these parameters are annealing temperature and time, extension time, Mg²⁺ and ATP concentration, pH, and the relative concentration of primers, templates and deoxyribonucleotides. After amplification, the resulting fragments can be detected by agarose gel electrophoresis followed by visualization with ethidium bromide staining and ultraviolet illumination.

In certain embodiments, the present disclosure further provides cells containing an expression vector, as described above, that contains a promoter; a coding sequence encoding a protein of interest in a reversed 3′-5′ orientation; a transcription termination sequence; and at least a first recombination site and a second recombination site flanking the coding sequence. In certain cases, the cells may contain additional vectors or genetic elements integrated into the cell genomes. In certain cases, the cells may be infected with a virus, e.g. adenovirus. In certain cases, the cells express a recombinase that recognizes the recombination sites in the expression vector.

### Methods

In certain aspects, the present disclosure provides a method of protein expression in a cell. In certain embodiments, the method involves transfecting a cell with a vector comprising a promoter, a coding sequence encoding a protein of interest, in which the coding sequence is in a reversed 3′-5′ orientation, a transcription termination sequence, and at least a first recombination site and a second recombination site flanking the coding sequence; exposing the vector to a recombinase, in which the recombinase recombines the first recombination site and the second recombination site to produce an inverted coding sequence and expresses the protein of interest.

In certain embodiments, the method involves 1) inserting a coding sequence encoding a protein of interest into a multiple cloning site such that the coding sequence is in the reversed 3′ to 5′ orientation relative to the promoter and flanked by at least two recombination sites, prior to transfecting the expression vector inserted with the coding sequence into a cell.

The method of transfection is well-known in the art and may comprise non-viral delivery systems or viral delivery systems. Non-viral delivery systems include but are not limited to DNA transfection methods. Here, transfection may include a process using a non-viral vector to deliver a gene to a target mammalian cell. Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), and combinations thereof.

Viral delivery systems include but are not limited to adenovirus vector, an adeno-associated viral (AAV) vector, a herpes viral vector, retroviral vector, lentiviral vector, baculoviral vector. In certain cases, viral based transformation protocols have been developed to introduce exogenous DNA to be subsequently integrated into the target cell's genome. In certain cases, the viral vectors are maintained episomally inside a cell. Other viral based vectors that find use include adenovirus derived vectors, HSV derived vectors, sindbis derived vectors, and retroviral vectors, e.g., Moloney murine leukemia viral based vectors, etc.

Other examples of vectors include ex vivo delivery systems—which include but are not limited to DNA transfection methods such as electroporation, DNA biolistics, lipid-mediated transfection, compacted DNA-mediated transfection). Certain features of the adenoviruses may be combined with the genetic stability of retroviruses/lentiviruses to transduce target cells that become capable of stably infect neighboring cells. Methods of viral delivery are well-known and are described in U.S. Pat. Pub. No. 2008/0175819, 2008/0050770, U.S. Pat. Nos. 6,303,362 and 7,045,344, disclosures of which are incorporated herein by reference.

In certain embodiments, the subject method comprises exposing the subject composition to a recombinase, such that the recombinase inverts the coding sequence between the first and the second recombination sites. The inverted coding sequence then becomes in the correct 5′ to 3′ orientation to enable transcription and translation of the expression product. In certain cases, the inverted coding sequence is incapable of subsequent recombination after being inverted into the 5′ to 3′ orientation.

Many recombinases may be used in the subject method. Two major families of site-specific recombinases from bacteria and unicellular yeasts have been described: the integrase or tyrosine recombinase family includes Cre, Flp, R, and λ integrase (Argos, et al., EMBO J. 5:433-440, (1986)) and the resolvase/invertase or serine recombinase family that includes some phage integrases, such as, those of phages φC31, R4, and TP901-1 (Hallet and Sherratt, FEMS Microbiol. Rev. 21:157-178 (1997)). For further description of suitable site-specific recombinases, see U.S. Pat. No. 6,632,672 and U.S. Patent Publication No. 2003/0050258, the disclosures of which are herein incorporated herein by reference in their entireties.

Action of the integrase upon these recognitions sites is unidirectional in that the enzymatic reaction produces nucleic acid recombination products that are not effective substrates of the integrase. This results in stable integration with little or no detectable recombinase-mediated excision, i.e., recombination that is “unidirectional”.

In certain embodiments, the recombinase used in the subject method is a unidirectional site-specific recombinase, such as a serine integrase. Serine integrases that may be useful for in vitro and in vivo recombination include, but are not limited to, integrases from phages φC31, R4, TP901-1, phiBT1, Bxb1, RV-1, A118, U153, and phiFC1, as well as others in the large serine integrase family (Gregory, Till and Smith, J. Bacteriol., 185:5320-5323 (2003); Groth and Calos, J. Mol. Biol. 335:667-678 (2004); Groth et al. PNAS 97:5995-6000 (2000); Olivares, Hollis and Calos, Gene 278:167-176 (2001); Smith and Thorpe, Molec. Microbiol., 4:122-129 (2002); Stoll, Ginsberg and Calos, J. Bacteriol., 184:3657-3663 (2002)). In addition to these wild-type integrases, altered integrases that bear mutations have been produced (Sclimenti, Thyagarajan and Calos, NAR, 29:5044-5051 (2001)). These integrases may have altered activity or specificity compared to the wild-type and are also useful for the in vitro recombination reaction and the integration reaction into the eukaryotic genome.

Alternatively, the subject method may employ the Cre recombinase/loxP recognition sites of bacteriophage P1 or the site-specific FLP recombinase of S. cerevisiae which catalyses recombination events between about 34 by FLP recognition targets (FRTs) (Karreman et al. (1996) NAR 24:1616-1624). A similar system has been developed using the Cre recombinase/loxP recognition sites of bacteriophage P1 (see PCT/GB00/03837; Vanin et al. (1997) J. Virol 71:7820-7826).

In certain embodiments, exposing a cell to a recombinase comprises turning on the expression of a recombinase encoded by the cell. The expression of a recombinase may be governed by methods known in the art. The recombinase may be encoded by a genomically integrated nucleic acid sequence or from a non-integrated extrachromosomal expression vector.

Briefly, in certain embodiments, the recombinase or a nucleic acid encoding the recombinase may be introduced into the host cell by transfection, e.g., as described above. Alternatively, the coding sequence for the recombinase may already be present in the host cell but not expressed, e.g., because it is under the control of an inducible promoter. In these embodiments, the inducible coding sequence may be present on another episomal nucleic acid, or integrated into the cell's genomic DNA. Representative inducible promoters of interest that may be operationally linked to the recombinase coding sequence include, but are not limited to: aracBAD promoter, the λ pL promoter, and the like, described above. In these embodiments, the step of providing the desired recombinase activity in the cell includes inducing the inducible promoter to cause expression of the desired recombinase.

Following production of the desired recombinase activity in a cell, the resultant cell is then maintained under conditions and for a period of time sufficient for the recombinase activity to mediate the inversion of the coding sequence into a 5′ to 3′ orientation. In certain cases, the host cell is maintained at a temperature of between about 20 and 40° C.

In cases where the subject method exposes the vector to a unidirectional site-specific recombinase is used, the coding sequence in the vector inverts into a translatable 5′ to 3′ orientation. As noted above, since the first and second recombination sites are recognizable by a unidirection recombinase to recombine in only one direction, the inverted coding sequence is incapable of subsequent recombination.

In certain cases, the method comprises transfecting into a cell an episomal circular expression vector comprising a third recombination site interposed between the first recombination site and the coding sequence and a fourth recombination site interposed between the second recombination site and the transcription termination region, in which the first recombination site and the second recombination site recombine in the presence of a recombinase and the third recombination site and the fourth recombination site recombine in the presence of a recombinase.

In certain cases, the first and second sites are the first pair of recombination sites and the third and fourth recombination sites are the second pair of recombination sites. In these embodiments, the subject method involves using a vector containing a first pair and a second pair of recombination sites that are incompatible with each other to undergo recombination.

The subject method employing a vector comprising a first pair and a second pair of recombination sites catalyzes a two-step recombination process that renders the inverted coding sequence incapable of subsequent recombination. As explained previously, this is because after a recombination event, excision of the intervening sequences between a pair of compatible recombination sites leaves the coding sequence flanked by incompatible recombination sites. For example, a first pair of sites may be loxP sites and the second pair may be lox27722 sites. Certain features of this recombination process are illustrated by a nonlimiting example in FIG. 2D and are explained above. In certain embodiments, the subject method comprises transfecting a vector comprising four recombination sites as set forth above and exposing the vector to a recombinase. Recombination ultimately leads to a coding sequence correctly oriented for production of an expression product and ensures stable orientation without subsequent excision or perpetual inversions of the coding sequence.

The subject method encompasses employing one or more embodiments of the vector described herein in order to express genes in a selected group of cells within a population. Since the expression of the trangene on the vector is dependent on recombination as described previously, and recombination depends on the presence of a recombinase, manipulating the availability of the recombinase in that selected group of cells is a means to control expression. One embodiment of selective gene expression is to transfect a vector as described herein in a population containing a recombinase-expressing cells. This allows only recombinase-expressing cells to express the transgene. Alternatively, the method may also encompass selecting a promoter that is specific for a subset of cells of interest to be used as the promoter driving the expression of the recombinase or transgene. In a related embodiment, cell-specific Cre transgenic mice, for example, may also be used for selective gene expression.

Not only can selective gene expression be carried out by taking advantage of distinct profiles of promoter elements or recombinase-expressing cell-types in transgenic mice but it can also be carried out based on cell to cell connection. In a multicellular organism, there are specific cell types that make contact with each other. The connection between cells (i.e. intercellular communication) may be utilized in the subject method to transfect or deliver the recombinase of interest from one cell to another.

For example, the subject method may encompass selectively targeting specific neurons for expression based on their topological organization (i.e. input and output targets). Neurons may be identified based on their projection patterns within the brain. As another example, the subject method allows the selective activation of a transgene in a subpopulation of excitatory pyramidal neurons that project their outputs to the amygdala. Similarly, other neuronal subpopulations can be effectively selected for gene expression based on their inputs and/or ouputs. This can greatly aid in mapping neuronal circuits in a complex brain structure.

One way to target selective neuronal population for gene expression is via the retrograde and anterograde transport mechanisms used by the neurons. For example, a large number of neuronal population “A” may be exposed to a vector carrying a transgene that is not expressed unless in the presence of a recombinase so that expression of the transgene is considered recombinase-dependent (e.g. Cre-dependent). Any of the vectors according to the subject composition may be used. The transgene may be flanked by one or more recombination sites. The promoter element driving the expression may also be flanked by one or more recombination sites. Accordingly, the transgene is expressed only when the necessary recombinase is delivered to these cells in population “A”. Selective delivery of the recombinase to a subpopulation of these cells may be carried out via the retrograde and anterograde transport of an upstream or downstream neuron, respectively. Details of how this method may be employed using the retrograde or anterograde transport machinery are set forth below.

If one would like to activate the transgene only in a subpopulation of cells in population “A” that projects to a region of interest, e.g. neuronal population “B”, one would transfect cells of population “B” (i.e. neurons downstream to “A”) with a recombinase-encoding vector. Particularly, the recombinase is engineered to be a fusion protein that interacts with the retrograde transport machinery. Once the neurons in population B receives such recombinase-encoding vector, the recombinase is expressed and retrogradely transported upstream only to a selected group of cells in population “A” that innervate cells in population “B”. As such, retrograde transport may used to selectively transport the recombinase protein to upstream neurons. Once the recombinase arrives in those selected cells in population “A”, it may then recombine the vector containing the trangene and allows transcription and translation of the transgene, in accordance with the method and composition described previously. In such a manner, one could control selective gene expression based on the type of projection destination the cells make.

For a retrograde-transported recombinase, the recombinase may be encoded in a viral vector and engineered to fuse to a retrograde transporting protein such as Rabis virus glycoprotein (RabiesG). Any other retrograde transporting protein may also be used.

On the other hand, in a case where cells in population “B” projects to cells in population “A”, and one is interested activating a transgene in such a subpopulation of cells in “A” that receives input from B, one would utilize components of the anterograde transport for selective gene expression. Population “B” will be transfected as above (i.e. injected) with the recombinase fused to an anterograde transporting protein so that the recombinase travels downstream to neurons in population “A” that are innervated by “B”. As such, only cells in “A” that are innervated by “B” would receive the recombinase resulting in subsequent recombination that activates gene expression.

For an anterograde-transported recombinase, the recombinase may be encoded in a viral vector and engineered to fuse to a retrograde transporting protein such as wheat germ agglutinin (WGA), Phaseolus vulgaris leucoagglutinin (PVL), or Cholera Toxin B (CTb).

A more tightly regulated gene expression method may also be carried out using a combination of any embodiment presented herein. For example, one or more different recombination sites may be used on the same or different vectors. E.g. the encoding sequence of a transgene may be in the reversed orientation as the described for the subject composition and flanked by one set of a recombination site while the promoter driving the transgene is also reversed and flanked by a different set of recombination sites. As such, two different and incompatible recombinases are required to activate the trangene. Depending on which group of cells are targeted for gene expression, each of the different recombinases may be transported upstream (retrograde) or downstream (anterograde) to the neuronal population transfected with the recombinase-encoding vector. Promoters of various strength may also be chosen to modulate the robustness of the transgene expression desired.

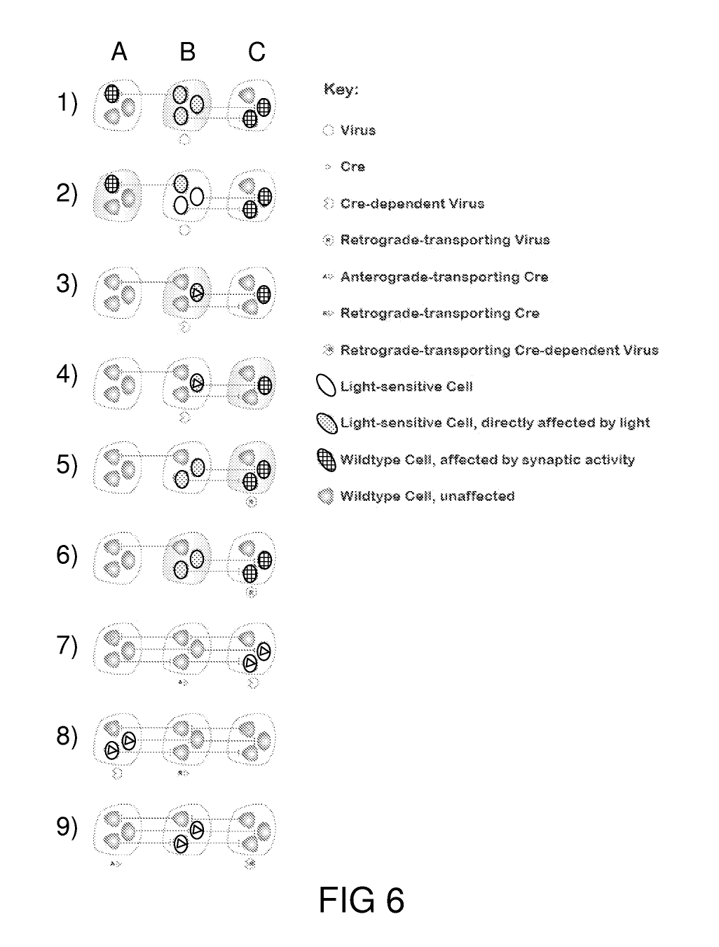

Using the various embodiments of the subject method, one could accomplish cell type- and circuit-specific gene expression. Various strategies for selective gene expression are presented in FIG. 6. The strategies are formulated for each of the nine exemplary networks of cells, each containing three populations of cells (A, B, and C) making different contacts with each other. Virus that is used in the exemplary networks below refers to virus carrying a vector encoding a light sensitive channel. In some networks, there is a cell population that has received light as a stimulus and is slightly shaded in the figure relative to the other two populations in the same network. Detailed explanation for strategies represented in FIG. 6 is set forth below.

In networks 1 and 2, population B is transfected with a virus, represented by the hexagon, carrying a vector that encodes a light sensitive channel (e.g. a microbial opsin). All cells in population B possess light sensitive channels and the cells they innervate, two in population C and one in population A are also affected by active synapses. In network 1, light is shone on population B, which activate all the light-sensitive cells in population B. This leads to all of their downstream neurons to be activated (checkered cells in populations A and C). In network 2, light is delivered only to population A. The one light-sensitive cell in population A and its axonal processes are activated accordingly. As such, the one cell in population B that is innervated by the stimulated cell in population A is activated.

In networks 3 and 4, population B is transfected with a Cre-dependent virus so the expression of the light-sensitive channel would be dependent on the presence of a Cre recombinase. As indicated by a triangle present in a cell in population B, Cre is only expressed in that one cell. As such, only that cell in population B expresses the light-sensitive channel and is light-sensitive. The cell it innervates in population C is then affected by synaptic activity. In network 3, light is delivered to population B, which causes the light-sensitive cell to release neurotransmitters. The released neurotransmitter would then lead to subsequent modulation of the downstream neuron in population C receiving input. Network 4 is different from network 3 in that light is delivered to population C as opposed to population B. As such, light is delivered in the downstream region to which the light-sensitive neuron is projecting. However, because of how the cells are connected, the modulation is the same for both networks 3 and 4.

As for networks 5 and 6, population C is transfected with a retrograde-transporting virus that carries a vector encoding a light sensitive channel. Two of the three cells make upstream connection with two cells in population B. As such, the light sensitive channel gets retrogradely transported upstream to those two cells in population B that innervate the two cells in population C. The two cells in population B then receive the light sensitive channel and become light-sensitive cells. The one cell in population C that does not make any connection with any cell in population B does not affect any cells in population B. Likewise, cells in population A are not transfected nor do they make any connection with a cell expressing any transgene so they do not become light sensitive. Similarly to networks 3 and 4 described above, although light is delivered to different cell populations in networks 5 and 6, the modulation outcome is the same.

In network 7, population C is transfected with a Cre-dependent virus so the expression of the light sensitive channel encoded by the viral vector would be dependent on the presence of the Cre recombinase. Since population B is transfected with anterograde-transporting Cre, all the cells in population B expresses Cre and transport Cre to their downstream neurons. Based on the connections between cells, one of the cells in population B transports Cre to one cell in population A and two others transport Cre to two cells in population C. Accordingly, the Cre that gets transported to two cells in population C would initiate the expression of the light sensitive channels in two of the cells in population C.

As for network 8, population B is transfected with retrogradely-transporting Cre so all the cells in population B transports Cre upstream. Two of the three cells transport Cre to two cells in population A and the third cell to a cell in population C. Since population A is transfected with Cre-dependent virus, the two cells that receive retrograde-transported Cre from population B are able to express the light sensitive channels and thus become light sensitive. One cell in population A does not have a retrograde-transported Cre because it is not upstream to any cells in population B and thus, does not express the light-sensitive channel. On the other hand, the cells in population C do not express any light sensitive channels regardless of whether there is a Cre recombinase or not because they do not possess a viral vector encoding a light sensitive channel.

Lastly, in network 9, population A is transfected with anterograde-transporting Cre and hence, the two cells that are upstream to cells in population B transport Cre to the cells in population B. One cell in population A is downstream to the cell in population B with which it makes a connection so it does not transport Cre to that cell in population B. Since population C is transfected with retrograde-transporting Cre-dependent virus, the two cells in population C transports Cre-dependent virus to the two upstream cells in population B. Since those two cells in population B also has Cre that have been transported antegradely from population A, the two cells in population B express the light sensitive channels.

Based on the strategies devised above for various types of networks, the subject method may be adapted to encompass many other permutations not listed above. The subject method may be modified so that a particular cell type or connection may be chosen to express the transgene of choice.

### Kit

Also provided by the present disclosure are kits for using the subject composition and for practicing the subject method, as described above. The subject kit contains a circular expression vector, comprising i) a promoter; ii) a multiple cloning site for inserting a coding sequence in a reversed 3′-5′ orientation; iii) a transcription termination sequence; and iv) at least a first recombination site and a second recombination site flanking the coding sequence, and instructions for using said vector.

In certain cases, the kit further comprises cells that are suitable for transfecting with the subject composition. In certain cases, the cells are suitable for the propagation of the circular expression vector. In additional embodiments, the cells contained in the subject kit may contain a recombinase or a nucleic acid sequence encoding a recombinase. The recombinase may recognize the recombination sites in the expression vector and catalyze an inversion of a intervening sequence between the recombination sites. The kit may further comprise an inducer to induce the expression of a recombinase in a cell.

In certain embodiments, the kit comprises one or more restriction enzymes. In other cases, the kit further comprises a map of the enclosed expression vector to aid a user in inserting a nucleic acid encoding an expression product of interest into a multiple cloning site.

In addition to above-mentioned components, the subject kit typically further includes instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the instructions, the kits may also include one or more control analyte mixtures, e.g., two or more control analytes for use in testing the kit.

### Utility

The subject invention finds use in a variety of applications, where such applications generally include, but not limited to, research applications, polypeptide synthesis applications, and therapeutic applications.

Examples of research applications in which the subject composition finds use include applications designed to characterize a particular gene with temporal and spatial controls. In such applications, the vector is employed to insert a gene of interest into a target cell, the coding sequence is inverted into a correct translatable orientation when desired, and the resultant effect of the expressed inserted gene on the cell's phenotype is observed. The ability to turn on gene expression when desired confers temporal and spatial control over experimental variables. In this manner, information about the gene's activity and the nature of the product encoded thereby can be deduced.

The subject composition may also be employed to identify and define DNA sequences that control gene expression, e.g. in a temporal (e.g. certain developmental stage) or spatial (e.g. particular cell or tissue type) manner. Yet another research application in which the subject composition finds use is in the identification and characterization of the results of gene expression studies. For example, a plurality of distinct vector targeted cells (or animals produced therefrom) are prepared in which the gene of interest is inserted into various targeted cells in an organism where a recombinase is expressed in different tissues or at different times. As such, the effects of gene expression on specific tissues or at different times may be compared. By plurality is meant at least two, where the number usually ranges from about 2 to 5000, usually from about 2 to 200. This plurality of vector targeted cells may be produced by introducing the vector in a plurality of cells or taking a collection of pretargeted cells that are homogenous with respect to the insertion site of the gene, i.e. progeny of a single targeted cell, and then introducing transposase into one or more of, but not all of, the constituent members of the collection.

The subject composition may also be used to study integration mutants, where a gene of interest is inserted randomly into the genome and the affects of this random insertion of the targeted cell phenotype are observed. One can also employ the subject vectors to produce models in which overexpression and/or misexpression of a gene of interest is produced in a cell and the effects of this mutant expression pattern are observed. One can also use the subject vectors to readily clone genes introduced into a host cell via insertional mutagenesis that yields phenotypes and/or expression patterns of interest. In such applications, the subject vectors are employed to generate insertional mutants through random integration of DNA. The phenotype and/or expression pattern of the resultant mutant is then assayed using any convenient protocol. The temporal and spatial control and the lack of leakiness may also allow transgenic animals and cells to survive prior to inverting the gene of interest into a translatable orientation if such gene expression turns out to be lethal.

In addition to the above research applications, the subject composition also finds use in the synthesis of polypeptides, e.g. proteins of interest. In such applications, a vector that includes a gene encoding the polypeptide of interest in combination with requisite and/or desired expression regulatory sequences, e.g. promoters, etc., (i.e. an expression module) is introduced into the target cell that is to serve as an expression host for expression of the polypeptide. Following introduction and subsequent stable integration into the target cell genome, the targeted host cell is then maintained under conditions sufficient for expression of the integrated gene. Once the transformed host expressing the protein is prepared, the protein is then purified to produce the desired protein comprising composition. Any convenient protein purification procedures may be employed, where suitable protein purification methodologies are described in Guide to Protein Purification, (Deuthser ed.) (Academic Press, 1990). For example, a lysate may be prepared from the expression host expressing the protein, and purified using HPLC, exclusion chromatography, gel electrophoresis, affinity chromatography, and the like.

Useful proteins that may be produced by the subject invention are, for example, enzymes that can be used for the production of nutrients and for performing enzymatic reactions in chemistry, or polypeptides which are useful and valuable as nutrients or for the treatment of human or animal diseases or for the prevention thereof, for example hormones, polypeptides with immunomodulatory activity, anti-viral and/or anti-tumor properties (e.g., maspin), antibodies, viral antigens, vaccines, clotting factors, enzyme inhibitors, foodstuffs, and the like. Other useful polypeptides that may be produced by the methods of the invention are, for example, those coding for hormones such as secretin, thymosin, relaxin, luteinizing hormone, parathyroid hormone, adrenocorticotropin, melanoycte-stimulating hormone, β-lipotropin, urogastrone or insulin, growth factors, such as epidermal growth factor, insulin-like growth factor (IGF), e.g. IGF-I and IGF-II, mast cell growth factor, nerve growth factor, glial cell line-derived neurotrophic factor (GDNF), or transforming growth factor (TGF), such as TGF-α or TGF-β (e.g. TGF-β1, β2 or β3), growth hormone, such as human or bovine growth hormones, interleukins, such as interleukin-1 or -2, human macrophage migration inhibitory factor (MIF), interferons, such as human α-interferon, for example interferon-αA, αB, αD or αF, α-interferon, γ-interferon or a hybrid interferon, for example an αA-αD- or an αB-αD-hybrid interferon, especially the hybrid interferon BDBB, protease inhibitors such as α₁-antitrypsin, SLPI, α₁-antichymotrypsin, C1 inhibitor, hepatitis virus antigens, such as hepatitis B virus surface or core antigen or hepatitis A virus antigen, or hepatitis nonA-nonB (i.e., hepatitis C) virus antigen, plasminogen activators, such as tissue plasminogen activator or urokinase, tumor necrosis factors (e.g., TNF-α or TNF-β), somatostatin, renin, β-endorphin, immunoglobulins, such as the light and/or heavy chains of immunoglobulin A, D, E, G, or M or human-mouse hybrid immunoglobulins, immunoglobulin binding factors, such as immunoglobulin E binding factor, e.g. sCD23 and the like, calcitonin, human calcitonin-related peptide, blood clotting factors, such as factor IX or VIIIc, erythropoietin, eglin, such as eglin C, desulphatohirudin, such as desulphatohirudin variant HV1, HV2 or PA, human superoxide dismutase, viral thymidine kinase, β-lactamase, glucose isomerase, transport proteins such as human plasma proteins, e.g., serum albumin and transferrin. Fusion proteins of the above may also be produced by the methods of the invention.

Furthermore, the levels of an expressed protein of interest can be increased by vector amplification (see Bebbington and Hentschel, “The use of vectors based on gene amplification for the expression of cloned genes in mammalian cells in “DNA cloning”, Vol. 3, Academic Press, New York, 1987). When a marker in the vector system expressing a protein is amplifiable, an increase in the level of an inhibitor of that marker, when present in the host cell culture, will increase the number of copies of the marker gene. Since the amplified region is associated with the protein-encoding gene, production of the protein of interest will concomitantly increase (Crouse et al., 1983, Mol. Cell. Biol., 3:257). An exemplary amplification system includes, but is not limited to, dihydrofolate reductase (DHFR), which confers resistance to its inhibitor methotrexate. Other suitable amplification systems include, but are not limited to, glutamine synthetase (and its inhibitor methionine sulfoximine), thymidine synthase (and its inhibitor 5-fluoro uridine), carbamyl-P-synthetase/aspartate transcarbamylase/dihydro-orotase (and its inhibitor N-(phosphonacetyl)-L-aspartate), ribonucleoside reductase (and its inhibitor hydroxyurea), ornithine decarboxylase (and its inhibitor difluoromethyl ornithine), adenosine deaminase (and its inhibitor deoxycoformycin), and the like.

In addition to the utilities described above, the subject invention may be used to deliver a wide variety of therapeutic nucleic acids. Therapeutic nucleic acids of interest include genes that replace defective genes in the target host cell, such as those responsible for genetic defect based diseased conditions; genes which have therapeutic utility in the treatment of cancer; and the like. Specific therapeutic genes for use in the treatment of genetic defect based disease conditions include genes encoding the following products: factor VIII, factor IX, β-globin, low-density protein receptor, adenosine deaminase, purine nucleoside phosphorylase, sphingomyelinase, glucocerebrosidase, cystic fibrosis transmembrane regulator, α-antitrypsin, CD-18, ornithine transcarbamylase, arginosuccinate synthetase, phenylalanine hydroxylase, branched-chain, α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, and the like. Cancer therapeutic genes that may be delivered via the subject vectors include: genes that enhance the antitumor activity of lymphocytes, genes whose expression product enhances the immunogenicity of tumor cells, tumor suppressor genes, toxin genes, suicide genes, multiple-drug resistance genes, antisense sequences, and the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Methods and Materials

The following methods and materials were used in the examples below.

Plasmids

The staggered loxP/lox2272 sites were designed using Vector NTI and synthesized by DNA 2.0 (Menlo Park, Calif.). The gene of interest (e.g. ChR2-EYFP) was cloned in between the loxP-lox2272 and lox2272-loxP sites using standard molecular cloning techniques. Briefly, ChR2-EYFP was PCR amplified using primers designed to append restriction sites to the 5′ and 3′ ends of ChR2-EYFP. Subsequently, the ChR2-EYFP PCR product and the construct carrying the staggered loxP/lox2272 sites were digested using restriction endonucleases. ChR2-EYFP is then subcloned into the loxP/lox2272-containing vector backbone via ligation.

The product containing loxP-lox2272-ChR2-EYFP (antisense)-loxP-lox2272 was then cloned into a vector containing the AAV2 ITRs. Also, polyA and WPRE DNA elements were also PCR amplified from templates and cloned into the AAV vector.

Virus Production

Recombinant virus vector were made using specific AAV serotypes depending on the target tissue (e.g. for gene delivery into the brain, AAV1, AAV2, or AAV5 were used). Briefly, the procedure is as follows.

To transfect one T-225 flask of 293T cells with vectors at a concentration of 1 μg/μl, the following protocol was used. For each flask, 63 μl AAV vector, 126 μl pDP1, pDP2, or pDP5 (depending on the desired serotype, for mosaic AAV, use 50:50 of each serotype plasmid), 510 μl 2M CaCl₂, and 1.45 mL distilled water were combined to create a DNA mixture. Next, the DNA mixture was combined with 2.15 mL of 2× HEPES buffered saline (50 mM HEPES, 1.5 mM Na₂HPO₄, 180 mM NaCl, pH 7.05) to create a transfection mixture. Lastly, the transfection mixture was added to 40.7 mL of Dulbecco's Modified Eagle's Medium (DMEM) with 10% fetal bovine serum (FBS). The cells were transfected by incubating the cells with the media mixed with the transfection mixture.

After fourteen hours post-transfection, media was removed and the cells washed once with 20 mL DMEM with 10% FBS (D-10). Flask was replaced with 40 mL of fresh D10. After seventy-two hours post-transfection, the cells were collected in 8.5 mL of Tris/NaCl solution (50 mM Tris, 150 mM NaCl) and frozen in dry ice/ethanol bath.

When the cells were ready to be used, the cells were thawed in 37° C. water bath and 500 μL 10% sodium deoxycholate monohydrate (Sigma Aldrich, D5670-5G, NaDOC) and 2 μL of Benzonase (Sigma Aldrich, E8263>=250 U/μL) were added. Incubation continued for 30 minutes at 37° C. 584 mg of NaCl were added to the cells. Incubation continued for 30 min at 56° C. The cells were frozen and thawed two more times before being loaded onto the following discontinuous iodixanol gradient: 60%-3 mL, 40%-3 mL, 25%-4 mL, 17%-7 mL, Virus-9 mL. Virus was spun in 70 Ti rotor for 90 minutes at 60,000 rpm and removed by taking 40% layer, diluted, concentrated in Amicon Concentrator with PBS-MK (1 mM MgCl₂, 2.5 mM KCl, PBS, pH 7.2), and filtered through 0.45 μm Acrodisc.

10 to 20 μl were analyzed on 10% acrylamide gel and stain with Comassie Blue Dye. Three bands, VP1 to VP3, were seen (data not shown).

Virus Delivery into the Brain

Recombinant AAV vectors were injected into brain areas of interest using stereotactic guidance. Surgeries were performed under aseptic conditions. For anaesthesia, ketamine (16 mg kg⁻¹ of body weight) and xylazine (5 mg kg⁻¹ of body weight) cocktail were injected intraperitoneally. Fur was sheared from the top of the animal's head and the head was placed in a stereotactic apparatus (David Kopf Instruments). A midline scalp incision was made and a 1-mm-diameter craniotomy was drilled. A glass micropipette was refilled with 3.0 μL of concentrated lentivirus solution using a programmable pump (PHD 2000, Harvard Apparatus) and 1 μL of lentivirus solution was injected at each site at a concentration of 0.1 mul min⁻¹.

Electrophysiology

Patch-clamp recordings in oocytes and neurons were carried out as previously described (Nagel, G. et al. Proc. Natl. Acad. Sci. USA 100:13940-13945 (2003), Nagel, G. et al. Science 296:2395-2398 (2002), and Boyden E. S. et al. Nature Neurosci. 8:1263-1268 (2005)). For whole-cell and cell-attached recording in cultured hippocampal neurons or acute brain slices, three intracellular solutions containing chloride were prepared: 4 mM chloride (135 mM K-gluconate, 10 mM HEPES, 4 mM KCl, 4 mM MgATP, 0.3 mM Na₃GTP, titrated to pH 7.2); 10 mM chloride (129 mM K-gluconate, 10 mM HEPES, 10 mM KCl, 4 mM MgATP, 0.3 mM Na₃GTP, titrated to pH 7.2); or 25 mM chloride (114 mM K-gluconate, 10 mM HEPES, 25 mM KCl, 4 mM MgATP, 0.3 mM Na₃GTP, titrated to pH 7.2). For cultured hippocampal neurons, Tyrode's solution was employed as the extracellular solution (125 mM NaCl, 2 mM KCl, 3 mM CaCl₂, 1 mM MgCl₂, 30 mM glucose, and 25 mM HEPES, titrated to pH 7.3). For preparation of acute brain slices, mice were killed 2 weeks after viral injection. Acute brain slices (250 mum) were prepared in ice-cold cutting solution (64 mM NaCl, 25 mM NaHCO₃, 10 mM glucose, 120 mM sucrose, 2.5 mM KCl, 1.25 mM NaH₂PO₄, 0.5 mM CaCl₂, 7 mM MgCl₂, and equilibrated with 95% O₂/5% CO₂) using a vibratome (VT1000S, Leica). Slices were incubated in oxygenated ACSF (124 mM NaCl, 3 mM KCl, 26 mM NaHCO₃, 1.25 mM NaH₂PO₄, 2.4 mM CaCl₂, 1.3 mM MgCl₂, 10 mM glucose, and equilibrated with 95%+O₂/5% CO₂) at 32° C. for 30 min to recover.

### Example 1

### Construction of the Vector

In cases where strategies for cell-specific protein expression depended on the use of endogenous cell-specific promoters, transcriptional activity based on specific endogenous promoters was low to moderate and was not adequate for certain applications. In applications that required the protein of interest to be expressed at high levels, the use of strong and ubiquitous promoters was desirable. Some examples of strong promoters included EF-1a, Ubiq, CAG, CMV, PGK, or the pan-neuronal promoters such as Synpasin I. However, employing a strong promoter in the context of a conventional floxed-stop construct might lead to transcriptional leakiness of the coding region. An exemplary of such floxed-stop construct is schematically illustrated as the Floxed Stop construct in FIG. 2A.

One approach to eliminate the transcriptional leakiness was to design a vector with the coding region positioned in an antisense orientation, as illustrated in the single-floxed inverse ORF (SIO) and double-floxed inverse ORF (DIO) of FIG. 2A. The coding region was not in the correct translational orientation unless a recombinase was present to invert the coding region.

After a coding region had been inverted by a recombinase, if the recognition sites for recombination were still present, such as the single pair of inverted loxP sites in SIO of FIG. 2A, perpetual inversion might occur, resulting in unstable expression of the coding region. However, if two pairs of staggered incompatible recognition sites were used to flank the antisense coding region, as illustrated in DIO of FIG. 2A, a two-step recombination process permanently flipped the coding region into the sense orientation relative to the promoter. The two-step recombination process with the DIO construct is schematically illustrated in FIG. 2D.

### Example 2

### Comparison of Constructs in HEK293 Cells

To investigate how the various constructs perform in terms of their leakiness and expression levels, Floxed-Stop, SIO, and DIO constructs were expressed in HEK293 cells in the absence and presence of Cre-recombinase. All constructs contained ChR2-EYFP as the gene of interest so the level of YFP signal was indicative of the level of expression.

Fluorescence micrographs of HEK293 cells expressing the various constructs in the presence or absence of Cre-recombinase were shown in FIG. 2B. The left panels represented cells expressing Cre, and hence the inversion was expected to enable translation of the coding region, resulting in expressing of ChR2-EYFP. The right panels represented cells without Cre so that no expression of ChR2-EYFP was expected to occur.

The top panels of FIG. 2B showed that cells transfected with the Floxed Stop construct had a very high YFP signal in the presence of Cre but also a relatively high YFP signal in the absence of Cre. This suggested that although Floxed Stop constructs gave very high expression of the gene of interest in the presence of Cre, the construct was leaky, leading to high background signals. Cells transfected with the SIO construct, as shown in the middle panels of FIG. 2B, had relatively low YFP signal whether or not Cre was expressed. Interestingly, cells transfected with the DIO construct showed moderately high YFP signal in the presence of Cre but virtually no YFP signal in the absence of Cre. This result indicated that DIO constructs eliminated the leaky expression associated with the Floxed Stop construct, while allowing stable expression of the coding region.

The level of detection sensitivity associated with the various constructs was determined using FACS. Fluorescence activated cell sorting (FACS) was used to quantify the population of cells exhibiting different levels of YFP fluorescence, transfected with one of the three constructs. The normalized amounts of cells were graphed against raw YFP fluorescence value as the x-axis, as shown in FIG. 2C. The graph representing the population of cells expressing Cre from each of the three constructs tested was overlaid with a graph of a corresponding population not expressing Cre.

FACS analysis suggested that the DIO construct enabled sensitized detection over background relative to the other constructs tested herein. For example, by comparing the graphs representing populations with or without Cre, the greatest difference between the two graphs in the area where the raw fluorescence value ranges from 10¹⁰ to almost 10³⁰, was observed in the bottom panel of FIG. 2C, where cells were transfected with the DIO constructs. This large differential between expression levels tightly regulated by Cre confirms that DIO constructs enable precise expression control of the gene of interest and facilitates detection over background.

### Example 3

### Expression of ChR2-EYFP in Neurons

A DIO construct was made using ChR2-EYFP as the gene of interest. The microbial light-sensitive proteins Chlamydomonas reinhardtii Channelrhodopsin-2 (ChR2-EYFP) allows the bidirectional control to turn the neurons on and off with high temporal precision and rapid reversibility. ChR2 is a monovalent cation channel that allows Na⁺ ions to enter the cell following an exposure to ˜470 nm blue light. Because of its fast temporal kinetics, on the scale of milliseconds, ChR2 allowed reliable trains of high frequency action potentials in vivo.

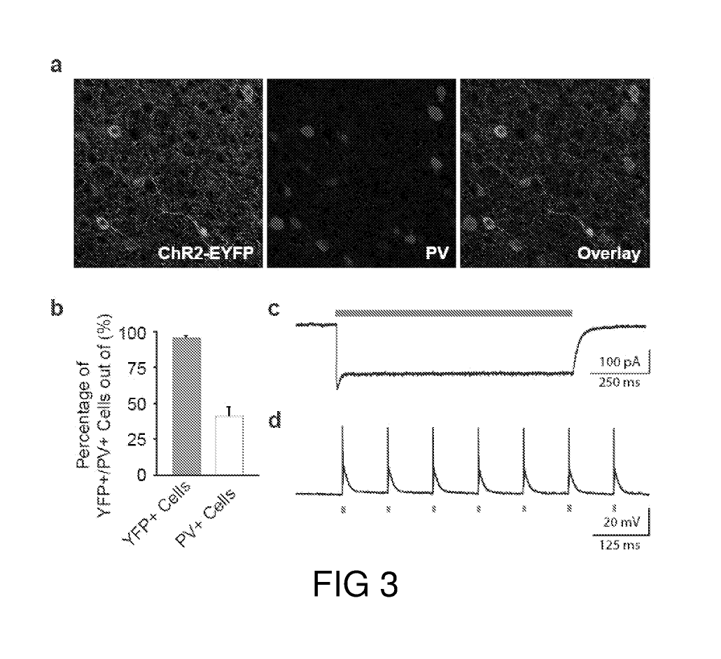

ChR2-EYFP directed ChR2 channel to be expressed with an enhanced yellow fluorescent protein as a tag. As shown in FIG. 3A, left panel, Cre-expressing hippocampal neurons transfected with the DIO construct containing ChR2-EYFP exhibited robust YFP fluorescence, an indication that the coding region had undergone inversion to be in the correct translation orientation. The middle panel of FIG. 3A showed fluorescence of cells expressing parvalbumin. Parvalbumin (PV) is present in GABAergic interneurons in the nervous system, predominantly expressed by chandelier and basket cells in the cortex. The two fluorescence channel monitoring ChR2-EYFP and PV were overlaid in the right panel, showing certain cells that expressed both ChR2-EYFP and PV. The percentage of cells that were either YFP positive or PV positive were calculated and shown as bar graphs in FIG. 3B. The fact that almost 100% of the cells were YFP positive indicated a stable inversion of the coding region of the DIO construct. The prevalence of YFP signal also suggested that two-step recombination process described in FIG. 2D was successful in preventing perpetual inversion subsequent to the coding region being in the correct translational orientation.

Further characterization of the DIO construct was carried out by investigating the functional expression of ChR2 in Cre-expressing neurons. Voltage-clamped neurons were illuminated by blue light (473 nm) for a constant period, indicated by the bar above the current trace in FIG. 3C. The exposure to light evoked an inward photocurrent, indicating that ChR2 channels were opened in response to the light stimulus. In another functional assay, neurons expressing ChR2 were illuminated with brief pulses of blue light, shown as dashes underneath the voltage trace in FIG. 3D. The whole-cell recording indicated that action potentials were evoked precisely with the application of the light stimulus. These results suggest that ChR2 expressed from the DIO construct were functional proteins that behaved predictably as ChR2 expressed from previously used constructs.

### Example 4

### Circuit-Specific Gene Expression Technology

The brain consists of numerous cell types interconnected and embedded within a heterogeneous tissue. Each cell type is characterized by a unique set of electrophysiological and biochemical characteristics and the assembly of several different cell types into a single circuit gives rise to computational units underlying diverse neurological functions ranging from basic motor control to complex emotional and cognitive functions.

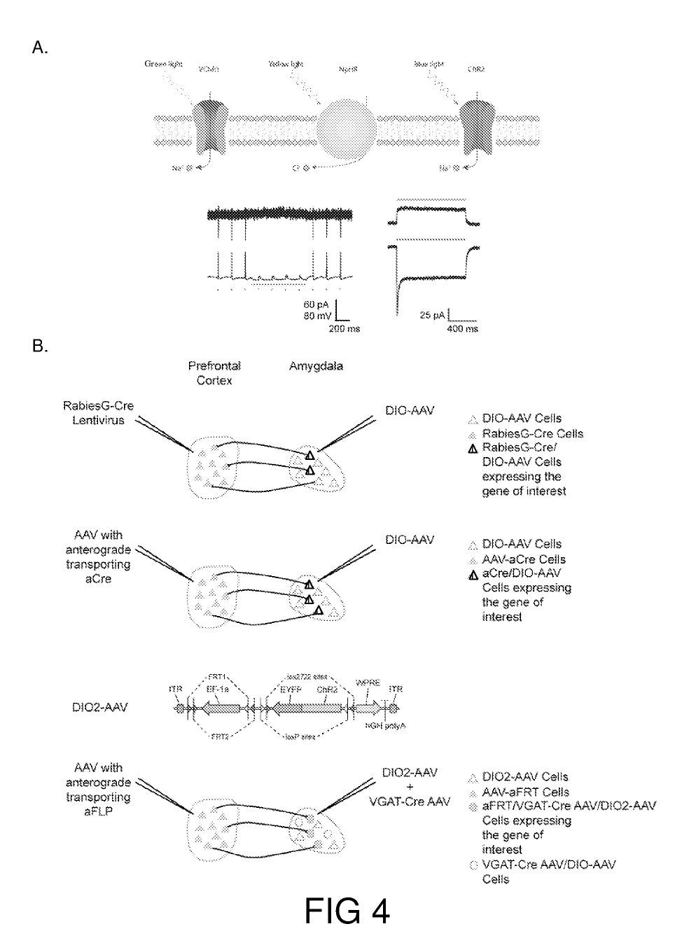

One way to interrogate the role of a specific cell type in a neural circuit may employ light-gated microbial opsins. These opsins can be used as neural activity regulators since they may be gated by exposure to brief flashes of blue or yellow light. Three microbial opsins are shown in FIG. 4, panel A. VChR1 and channelrhodopsin-2 (ChR2) are capable of exciting neurons using green and blue light respectively and halorhodopsin (NpHR) is capable of inhibiting neural activity upon exposure to yellow light. The bottom left of panel A shows two voltage traces of NpHR (top trace) and ChR2 (bottom trace), demonstrating the ability of light flashes to mediate bidirectional optical control of neural activity. In bottom left of panel A, bars or dashes underneath the trace represent flashes of light for NpHR or ChR2 respectively. In the bottom right of panel A, yellow and blue light evoked outward and inward current respectively in a neuron expressing NpHR and ChR2.

The use of these microbial opsins such as ChR2 and NpHR allow the application of many existing genetic techniques to render specific sets of neurons light-sensitive, therefore allowing the control of the function of a set of genetically identical neurons within the heterogeneously populated brain tissue, without affecting nearby cells. One example of applying the method described in the present disclosure is set forth below.

One purpose may encompass the use of genetically-encoded neural activity regulators to perturb a selected population of neurons in the heterogeneously populated brain without affecting adjacent cells. To achieve strong levels of ChR2 and NpHR expression in specific neuron populations, a Cre-inducible Adeno-associated virus (referred to herein as DIO-AAV) was developed based on a system to decouple cell-specificity from transcriptional-strength. In this system, the DIO-AAV vector carries a strong ubiquitous promoter and an inactivated open reading frame (ORF) in accordance with the embodiments described previously in the present disclosure. When the virus is delivered into a transgenic animal expressing the Cre recombinase under a cell specific promoter, the ORF becomes activated in Cre-expressing cells (FIG. 2, panel D). In addition to utilizing this expression system, various approaches may be incorporated to restrict gene expression to the circuit of interest. The following three approaches provide solutions to target gene expression with cell type- and circuit-specificity by leveraging unique properties of viral and plant/microbial proteins to transport across neurons in a retrograde or anterograde manner. These systems are also generally applicable to a variety of mammalian animal models not limited to mice.

Exemplary Approach I: Combining Retrograde-Transporting Viruses and DIO-AAV to Enable Gene Expression in Specific Sets of Neurons Based on Projection Patterns (Top of FIG. 4, Panel B).

As the different downstream regions may be involved in different activities, it may be useful to be able to restrict gene expression not based on the biochemical marker but also based on their projection destination. In the prefrontal cortex, for example, excitatory pyramidal neurons can be divided into different groups based on their projection destination (e.g. Nucleus accumbens and amygdala) (Gorelova, N. et al. Neuroscience 76:689-706 (1997); Rosenkranz, J. A. et al. J Neurosci 23: 11054-11064 (2003)). Although all excitatory neurons may be targeted using the excitatory neuron-specific CaMKIIa promoter (Aravanis, A. M., et al. J Neural Eng 4:S143-156 (2007)), it is may be useful to target only the pyramidal neurons that are exclusively projecting to the brain region of interest. With such control targeting, only the cortical activity that affects the downstream region of interest is altered during a behavior test where the activity of excitatory neurons in the prefrontal cortex is changed.

In order to achieve selective gene expression on neurons of a specific projection destination, a Cre-carrying lentivirus pseudotyped with the Rabies virus glycoprotein (RabiesG) was developed. The RabiesG-pseudotyped lentivirus is known to endow retrograde transport properties to the lentivirus so that the virus can enter a cell through its axon and travel back to the cell's nucleus to complete transduction (Watson, D. J. et al. Mol Ther 5:528-537 (2002)). Using this system, the RabiesG-pseudotyped Cre lentivirus may be delivered downstream where the axons termini are located and DIO-AAV may be delivered to the brain region where the cell bodies are located. The Cre lentivirus would travel back to the cell bodies and activate DIO-AAV only in cells that are projecting to the injection site of the Cre lentivirus. Unlike DIO-AAV, which depends on the availability of cell-specific Cre transgenic mice, this combined system can be applied in any mammalian animal model.

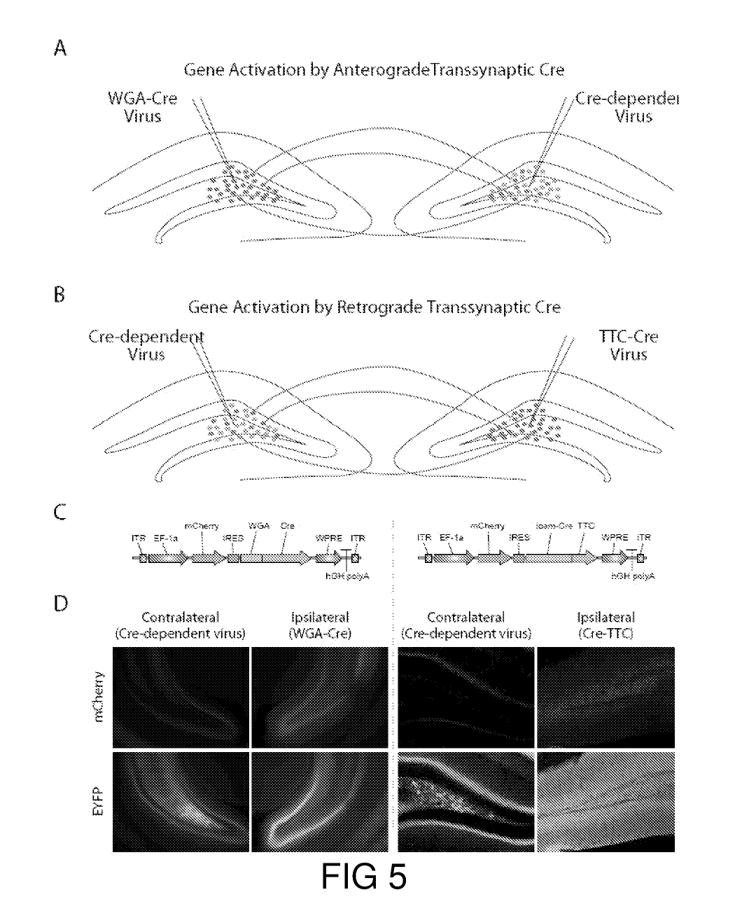

More detail of retrograde targeting strategy is presented in FIG. 5 panels B, C, and D. Panel B shows a schematic illustrating a strategy for targeting hippocampal dentate gyms neurons sending projections to the contralateral dentate gyms. Retrograde (Cre-TTC) transsynaptic Cre virus carrying the construct shown in right of panel C in FIG. 5 is injected into the ipsilateral dentate gyms. A Cre-dependent virus is injected into the contralateral dentate gyms of the same animal.

Upon expression, Cre-TTC are transynaptically transported to the upstream neurons to activate Cre-dependent gene expression in the targeted neurons. Fluorescence images at the right of panel D in FIG. 5 shows activation of Cre-dependent gene expression in the contralateral dentate gyms via transsynaptic accumulation of Cre, although no TTC-Cre is injected into contralateral dentate gyms.

In addition to the retrograde-transporting RabiesG-pseudotyped lentiviral vectors, recombinant Herpes Simplex Virus-1 (HSV-1) vectors can also be used to achieve retrograde gene expression in neurons projecting to the vector injection site.

Exemplary Approach 2: Engineer an Anterograde-Transporting Cre Recombinase to Achieve an Anterograde-Activating Cre-Inducible Expression Systems (Middle of FIG. 4, Panel B).

The RabiesG-pseudotyped Cre lentivirus gives us the ability to control gene expression in upstream projection neurons through retrograde gene transfer. However, it is also important to be able to target gene expression to downstream neurons. For example, in regions such as the prefrontal cortex which receive innervations from numerous upstream regions (Gigg, J. et al. Hippocampus 4:189-198 (1994); Akirav, I. et al. Neural Plast 2007:30873 (2007)), one may want to be able to modulate cells that are innervated by the amygdala independently from the cells that are innervated by the hippocampus. In this way, the prefrontal cortex's role in processing emotional input from the amygdala may be distinguished from memory input coming from the hippocampus.

To achieve anterograde-specific gene expression, the Cre recombinase is to be engineered with anterograde transport properties. This can be accomplished by engineering a fusion of Cre with an anterograde-transporting protein (referred to herein as aCre) such as the wheat germ agglutinin (WGA) (Fabian, R. H. et al. Brain Res 344:41-48 (1985)), Phaseolus vulgaris leucoagglutinin (PVL) (Cucchiaro, J. B. et al. J Electron Microsc Tech 15:352-368 (1990)), and the (CTb) (Dederen, P. J. et al. Histochem J 26:856-862 (1994)). The recombinant versions of these proteins have been historically used as neural tracers. In this approach, instead of using recombinant proteins, an AAV vector carrying aCre is generated. Similar to how the RabiesG-pseudotyped Cre lentivirus is delivered in exemplary approach 1 presented above, the aCre AAV vector is stereotactically delivered upstream and the DIO-AAV vector downstream. The upstream cells would begin to produce aCre proteins, which would then be transported through the axon to the target site and secreted to the postsynaptic neuron to activate DIO-AAV.

More detail of anterograde targeting strategy is presented in FIG. 5 panels A, C, and D. Panel A shows a schematic illustrating a strategy for targeting hippocampal dentate gyms neurons receiving projections from the contralateral dentate gyms. Anterograde (WGA-Cre) virus carrying the construct shown in left of panel C in FIG. 5 is injected into the ipsilateral dentate gyms. A Cre-dependent virus is injected into the contralateral dentate gyms of the same animal.

Upon expression, WGA-CRE are transynaptically transported to the downstream neurons to activate Cre-dependent gene expression in the targeted neurons. Fluorescence images at the left of panel D in FIG. 5 shows activation of Cre-dependent gene expression in the contralateral dentate gyms via transsynaptic accumulation of Cre, although no WGA-Cre is injected into contralateral dentate gyms.

In certain cases, anterograde transporting properties may be enhanced by appending an exporting signal such as the N-terminal signal peptide from Icam to the Cre fusion protein to facilitate membrane trafficking of the fusion proteins.

In the approach disclosed herein, the levels of protein expression may fine tuned to accommodate various cellular and experimental systems. This can be done through a combination of promoter choice, codon optimization, and the use of destabilizing signal peptides. Since transcriptional strength is decoupled from the transcriptional specificity in the inducible expression system disclosed herein, one may tune down the Cre expression level without compromising the expression level of the protein of interest, such as ChR2, NpHR and a varieties of genetically-encoded activity sensors and markers. This method and system can also be generally applied in all animal models without dependence on transgenic mice.

Exemplary Approach 3: Engineer a Combinatorial Anterograde-/Retrograde-Activating System Using Cre and Flp to Achieve Cell Type- and Circuit-Specific Expression of Neural Activity Modulators and Sensors (Bottom of FIG. 4, Panel B).

The cells in a given downstream region receiving input from the same upstream region can be quite diverse, and are thought to be involved in different types of behaviors or different stages of the same behavior. For example, the excitatory neurons from the prefrontal cortex projects to the basal lateral and central nucleus of the amygdale. Some prefrontal cortical neurons selectively project to the inhibitory intercalated cells and others project to excitatory neurons in the amygdala. During the presentation of a fear stimulus, the amygdalar inhibitory and excitatory neurons are recruited by the prefrontal cortex for different phases of the fear response such as the acquisition, extinction, and relapse of fear conditioned responses (Herry, C., et al. Nature 454:600-606 (2008)). Therefore, it may be useful to control the gene expression of activity modulators (e.g. ChR2 and NpHR) and sensors (e.g. GFP and GCamp2) in specific types of downstream neurons such as the amygdalar inhibitory neurons that receive input from the prefrontal cortex and the amygdalar excitatory neurons that project to the prefrontal cortex. With such control over gene expression, the relevant subset of neurons may be modulated or monitored during a behavior experiment to study their specific involvement.

Cell type- and circuit-specific gene expression may be accomplished using two incompatible recombinases such as Cre and Flp. In this vector system (referred to as DIO2-AAV), the DIO-AAV vector will be reengineered so that the promoter and the ORF are both in the reverse orientation (FIG. 4, panel B). The promoter will be flanked with two sets of incompatible FRT sites and the ORF will be flanked by two sets of incompatible lox sites as in the original DIO-AAV design. In an experiment where ChR2 is specifically expressed in the inhibitory neurons of the amygdala that are innervated by prefrontal cortical cells, an AAV vector carrying aFlp (Flp engineered to have anterograde transport properties) is injected to the prefrontal cortex. In the amygdale, a mixture of AAV carrying Cre under the inhibitory neuron-specific VGAT promoter and DIO2-AAV carrying the transgene of interest are injected. Following viral delivery, although all DIO2-AAV will non-preferentially enter into all cells in the amygdala, only those cells that are have both Flp and Cre will be able to express the transgene of interest. Since Hp will be specifically coming from the prefrontal cortical projections and Cre expression will be tightly regulated by the VGAT promoter, only those inhibitory neurons postsynaptic to the prefrontal cortical cells would express the transgene of interest. This exemplary method and system is also generally applicable in all mammalian animal models.

This approach utilizes the inhibitory neuron-specific VGAT promoter to drive Cre expression. However, in alternate embodiments, cell-specific Cre expression may also be driven by a bacterial artificial chromosome (BAC) transgenic construct.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A circular expression vector, comprising:
a promoter;
a coding sequence encoding a protein of interest, wherein the coding sequence is in a reversed 3′-5′ orientation;
a transcription termination sequence; and
at least a first recombination site and a second recombination site flanking the coding sequence.

2. The vector of claim 1, wherein said coding sequence when inverted by a recombinase is incapable of subsequent recombination.

3. The vector of claim 1, wherein the first recombination site is an attP site and the second recombination site is an attB site.

4. The vector of claim 3, wherein said attP site and said attB site recombine in the presence of a unidirectional, site-specific recombinase.

5. The vector of claim 4, wherein said unidirectional, site-specific recombinase is phiC31 integrase.

6. The vector of claim 1, wherein the vector further comprises a third recombination site interposed between said first recombination site and said coding sequence and a fourth recombination site interposed between said second recombination site and said transcription termination region, wherein said first recombination site and said second recombination site recombine in the presence of a recombinase and said third recombination site and said fourth recombination site recombine in the presence of a recombinase.

7. The vector of claim 6, wherein said first recombination site and second recombination site are LoxP sites and said third recombination site and fourth recombination site are Lox2722 sites.

8. The vector of claim 6, wherein said recombinase is Cre recombinase or Flp recombinase.

9. The vector of claim 1, wherein said circular expression vector is an episomal vector.

10. The vector of claim 7, wherein said episomal vector is an adeno-associated vector.

11. The vector of claim 7, wherein said episomal vector is an EBNA-1 based episomal vector.

12. A method of expressing a protein of interest in a cell, comprising:
transfecting a cell with an episomal circular expression vector, comprising:
a promoter;
a coding sequence encoding a protein of interest, wherein the coding sequence is in a reversed 3′-5′ orientation;
a transcription termination sequence; and
at least a first recombination site and a second recombination site flanking the coding sequence; and
exposing the vector to a recombinase,
wherein said recombinase recombines said first recombination site and said second recombination to produce an inverted coding sequence and expression of the protein of interest.

13. The method of claim 12, wherein said exposing the vector to a recombinase renders said inverted coding sequence incapable of subsequent recombination.

14. The method of claim 12, wherein said first recombination site is an attP site and said second recombination site is an attB site.

15. The method of claim 14, wherein said attP site and said attB site recombine in the presence of a unidirectional, site-specific recombinase.

16. The method of claim 15, wherein said unidirectional, site-specific recombinase is phiC31 integrase.

17. The method of claim 12, wherein said episomal circular expression vector further comprises a third recombination site interposed between said first recombination site and said coding sequence and a fourth recombination site interposed between said second recombination site and said transcription termination region, wherein said first recombination site and said second recombination site recombine in the presence of a recombinase and said third recombination site and said fourth recombination site recombine in the presence of a recombinase.

18. The method of claim 17, wherein said first recombination site and said second recombination site are LoxP sites and said third recombination site and said fourth recombination site are Lox2722 sites.

19. The method of claim 17, wherein said recombinase is Cre recombinase or Flp.

20. The method of claim 18, wherein said episomal vector is an adeno-associated vector.

21. The method of claim 18, wherein said episomal vector is an EBNA-1 based episomal vector.

22. A kit comprising:
a circular expression vector, comprising
i) a promoter;
ii) a multiple cloning site for inserting a coding sequence in a reversed 3′-5′ orientation;
iii) a transcription termination sequence; and
iv) at least a first recombination site and a second recombination site flanking the coding sequence, and
instructions for using said vector.

23. The kit of claim 22, wherein said kit further comprises cells.

24. The kit of claim 23, wherein said cells express a recombinase.

25. The kit of claim 24, wherein said recombinase is Cre recombinase, Flp, or phiC31 integrase.
